(19) United States
(12) Patent Application Publication
(10) Pub. No.: US 2010/0040694 A1
(43) Pub. Date: Feb. 18, 2010

(54) LOW-MOLECULAR WEIGHT, WATER-SOLUBLE CHITOSAN NANOPARTICLE FOR GENE DELIVERY WITH FOLIC ACID CONJUGAED THERETO AS TARGET LIGAND AND PREPARATION METHOD THEREOF

(75) Inventors: Jae Woon Nah, Jeollanam-do (KR); Teok Rae Jung, Gyeonggi-do (KR); Mi Kyeong Jang, Jeollanam-do (KR); Dong Gon Kim, Jeollanam-do (KR); Sun Heang Heo, Jeollanam-do (KR)

Correspondence Address: LUCAS & MERCANTI, LLP, 475 PARK AVENUE SOUTH, 15TH FLOOR, NEW YORK, NY, 10016, US

(73) Assignee: KITTO LIFE, Pyongtaeg-si Gyeonggi-do (KR)

(21) Appl. No.: 12/514,351

(22) PCT Filed: Nov. 14, 2007
(86) PCT No.: PCT/KR2007/005711

§ 371 Date: May 11, 2009

(30) Foreign Application Priority Data
Nov. 14, 2006 (KR) 10-2006-0112418

(51) Int. Cl. A61K 9/14; C08B 37/08; A61K 31/722

(52) U.S. Cl. 424/489; 536/20; 514/55; 977/773

(57) ABSTRACT

Disclosed are low-molecular weight, water-soluble chitosan nanoparticles with folic acid conjugated thereto as a target ligand and a preparation method thereof. The nanoparticles can be simply prepared since the strong reactivity of the chitosan allows folic acid to be readily introduced thereinto. Also, the folic acid-conjugated, low-molecular weight, water-soluble chitosan nanoparticles can be useful as gene carriers because they are of low or zero-toxicity, have sizes suitable for use as gene carriers, can readily form complexes with DNA, allow high gene expression rates, and are excellent in targeting tumor cells which are rich in folic acid receptors.

### TECHNICAL FIELD

The present invention relates to low-molecular weight, water-soluble chitosan nanoparticles for gene delivery to which folic acid is conjugated as a target ligand. Also, the present invention is concerned with a method of preparing the nanoparticles.

### BACKGROUND ART

Gene therapy is the insertion of therapeutic genes into target cells and tissues to treat a disease, particularly hereditary diseases, in which a defective mutant allele is replaced with a functional one to express a functional protein. Having excellent selectivity compared to general drug therapy, gene therapy can be applied to the treatment of diseases for a prolonged period of time at higher curing rates and efficiency. Gene therapy has been developed to remove the causes of diseases, rather than merely to target the symptoms of diseases. For effective gene therapy, there is a need for gene delivery technology by which a gene of interest is introduced into a target cell and is expressed therein at a high rate.

For use in gene therapy, gene carriers must be of low or zero toxicity and must be able to deliver a gene of interest into a target cell with high selectivity and effectiveness. Gene carriers are typically classified as either viral or non-viral carriers.

Examples of viral gene carriers include retroviruses (RV), adenoviruses (AV), and adeno-associated viruses (AAV). These gene carriers are excellent in expression rate and persistency, but entail the risk of inducing immunity, causing toxicity and accumulating in the body [R. S. Kevin, Gene therapy, 34, 247-268 (2003); E. Marshall, Gene therapy's growing pains, Science, 269, 1050-1055 (1995)]. For instance, an 18-year old youth died in 1999 during gene therapy using an adenovirus as a gene carrier at the University of Pennsylvania. The FDA and the NIH consequently prohibited all clinical experiments involving gene therapy using adenoviruses.

With the occurrence of the accident, a lot of attention has been paid to non-viral gene carriers.

As non-viral gene carriers, cationic lipids or polymers are typically used. They form stable complexes with anionic DNA via ion bonds so as to deliver DNA into cells. Compared to viral gene carriers, non-viral gene carriers, such as cationic liposomes, enjoy the advantages of higher biodegradability, lower toxicity and greater non-immunogenicity, and greater convenience for use, but suffer from the disadvantage of lower delivery efficiency [K. Morimoto, M. Nishikawa, S. Kawakami, T. Nakano, Y. Hattori, S. Fumoto, F. Yamashita and M. Hashida, Molecular weight-dependent gene transfection activity of unmodified and galactosylated polyethylenimine on hepatoma cells and mouse liver, Mol. Therapy, 7, 254-261 (2003)].

Therefore, there is a need for a non-viral gene carrier that is highly effective in gene delivery.

Chitosan is a biopolymer formed by −1,4 linkage of pyranose monomers of glucosamine, having over 5,000 residues of glucosamine. Its molecular weight is over one million. As a biopolymer belonging to polysaccharides having polycations, chitosan is extracted from aquatic products such as Crustaceans, like crab or shrimp, and squid. Having a structure similar to that of cellulose, chitosan is highly biocompatible, avoiding rejection by immune reaction. Recently, chitosan has found applications in the medical industry. After FDA approval for food, chitosan has recently arisen as one of the most important materials useful in bioengineering and biomedical industries in the 21^{st} century.

Particularly, chitosan, ranging in molecular weight from 20,000 to 100,000, is known to show potent physiological activity and is a research target of great interest for use in a variety of fields including health foods, food and beverages, cosmetics, sanitation, pharmaceuticals, medicines, and medical supplies.

Although it has promising characteristics and merits, however, chitosan is difficult to successfully apply in practice because it is highly water-insoluble due to the strong hydrogen bonds between neighboring chitosan molecules. Conventionally, organic acids, such as lactic acid, acetic acid, propionic acid, formic acid, ascorbic acid, and tartaric acid, and inorganic acids such as hydrochloric acid, nitric acid, sulfuric acid, etc., are used to dissolve chitosan in water, but act as obstacles in the application of chitosan to the body. Korean Pat. No. 441270, issued to the present inventors, disclosed a surprising water-soluble chitosan, devised to overcome the above-mentioned problems.

In detail, as disclosed in Korean Pat. No. 441270, pure, water-soluble, free amine chitosan can be prepared by 1) treating an organic or inorganic acid salt solution of chitosan oligosaccharide solution with trialkyl amine, 2) adding an organic solvent to the solution to remove the organic acid or inorganic acid that is linked with chitosan in the form of a trialkyl amine salt, and recovering chitosan oligosaccharide that is free of organic or inorganic salt, 3) treating the acid-free chitosan oligosaccharide solution with an inorganic acid, followed by purification through an activated carbon/ion exchange column to give water-soluble chitosan having a molecular weight of 1,000 to 100,000 Da.

Such low-molecular weight, water-soluble chitosan is non-toxic, and thus biocompatible. The high water solubility makes it possible to use distilled water as an injection solvent that is non-toxic. Also, it is degraded by lysozyme, and undergoes no rejection by immune reaction. Having these advantages, chitosan can be a promising candidate as a gene carrier for the delivery of therapeutic genes into cells. However, chitosan itself is unable to target specific cells, and may influence normal cells as well.

Folic acid, a conjugate of glutamic acid residues with pteroic acid, plays a variety of roles in biosynthetic reactions. For example, folic acid, or folate, is essential for nucleic acid synthesis, and hence cell division. Also, folate derivatives are substrates that are involved in a number of single-carbon-transfer reactions and amino acid metabolism. Folic acid is an essential nutrient that is involved in nucleic acid synthesis, energy generation and erythrocyte maturation, playing a particularly important role in cell proliferation and growth. Accordingly, a great variety of tumor cells have folic acid receptors (FR) in abundance so as to have great affinity with folic acid because they need a large amount of various nutrients and require high metabolic rates for their rapid growth and proliferation. Folic acid can act as a marker for tumors because it is distributed in a low density throughout normal tissues, in contrast to tumor cells [P. Caliceti, S. Salmaso, A. Semenzato, T. Carofiglio, R. Formasier, M. Fermeglia, M. Ferrone, and S. Pricl, Bioconjugate Chem., 14, 899 (2003); S. Wang, R. J. Lee, C. J. Mathias, M. A. Green, and P. S. Low, Bioconjugate Chem., 7, 56 (1996)].

Leading to the present invention, intensive and thorough research into a gene carrier for delivering a gene into specific tumor cells at high efficiency and in safety, conducted by the present inventors, resulted in the finding that the conjugation of low-molecular weight, water-soluble chitosan with folic acid forms globular core-shell nanoparticles which are suitable for use as gene carriers in terms of size, association with DNA, gene expression efficiency, and feasibility, to thus target folic acid receptors, which are abundantly distributed throughout tumor cells.

### DISCLOSURE

### Technical Problem

It is an object of the present invention to provide a conjugate compound of low-molecular weight, water-soluble chitosan with folic acid.

It is another object of the present invention to provide a low-molecular weight, water-soluble chitosan nanoparticle for use as a gene carrier, with folic acid conjugated as a target ligand thereto.

It is a further object of the present invention to provide a water-soluble chitosan nanoparticle complex with a gene encapsulated therein.

It is still a further object of the present invention to provide methods of preparing the folic acid-conjugated, low-molecular weight, water-soluble chitosan nanoparticle and the water-soluble chitosan nanoparticle-gene complex.

### Technical Solution

In order to accomplish the above-mentioned objects, the present invention provides low-molecular weight, water-soluble chitosan nanoparticles to which folic acid is conjugated as a target ligand and a method of preparing the same.

### ADVANTAGEOUS EFFECTS

The low-molecular weight, water-soluble chitosan nanoparticles with folic acid conjugated thereto as a target ligand in accordance with the present invention can be simply prepared since the strong reactivity of the chitosan allows folic acid to be readily introduced thereinto. Also, the folic acid-conjugated, low-molecular weight, water-soluble chitosan nanoparticles can be useful as gene carriers because they are of low or zero-toxicity, have sizes suitable for use as gene carriers, can readily form complexes with DNA, allow high gene expression rates, and are excellent in targeting tumor cells which are rich in folic acid receptors.

### DESCRIPTION OF DRAWINGS

FIG. 1 shows FT-IR spectra of folic acid-conjugated, low-molecular weight, water-soluble chitosan nanoparticles in accordance with an embodiment of the present invention,

FIG. 2 shows ¹H NMR spectra of folic acid-conjugated, low-molecular weight, water-soluble chitosan nanoparticles in accordance with an embodiment of the present invention,

FIG. 3 is a graph showing particle sizes and size distributions of folic acid-conjugated, low-molecular weight, water-soluble chitosan nanoparticles in accordance with an embodiment of the present invention

FIG. 4 is a TEM photograph of folic acid-conjugated, low-molecular weight, water-soluble chitosan nanoparticles in accordance with an embodiment of the present invention,

FIG. 5 is a photograph showing the mobility of complexes of folic acid-conjugated, low-molecular weight, water-soluble chitosan nanoparticles in accordance with an embodiment of the present invention with DNA,

FIG. 6 shows expression rates of folic acid-conjugated, low-molecular weight, water-soluble chitosan nanoparticle-gene complexes at pH 6.2 in accordance with an embodiment of the present invention,

FIG. 7 is a graph showing cell viability at pH 6.2 when cells are treated with folic acid-conjugated, low-molecular weight, water-soluble chitosan nanoparticles in accordance with an embodiment of the present invention.

### BEST MODE

In accordance with an aspect thereof, the present invention provides a conjugate compound of the low-molecular weight, water-soluble chitosan represented by the following Chemical Formula 1 and folic acid

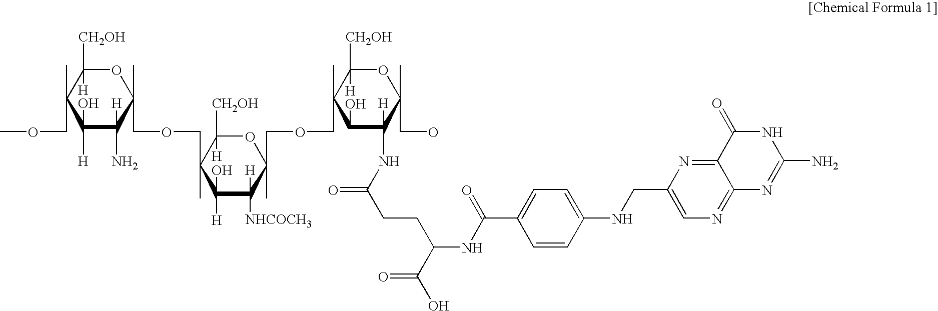

In accordance with another aspect thereof, the present invention provides water-soluble chitosan nanoparticles as gene carriers, which comprise low-molecular weight, water-soluble chitosan with folic acid conjugated thereto.

The water-soluble chitosan nanoparticles for the delivery of genes in accordance with the present invention are constructed by grafting hydrophobic folic acid to the chain of low-molecular weight, water-soluble chitosan. Preferably, the low-molecular weight, water-soluble chitosan and the folic acid are mixed in a weight ratio of 90˜110:0.5˜1.5. In this weight range, the nanoparticles can form self-aggregates suitable for use in transferring genes.

Consisting of hydrophilic low-molecular weight chitosan and hydrophobic folic acid, the water-soluble chitosan nanoparticles for the delivery of genes in accordance with the present invention show properties of amphophilic compounds. In an aqueous solution, the nanoparticle forms an aggregate with a hydrophobic core surrounded by a hydrophilic shell. Taking a form of globular core-shell structure, the low-molecular water-soluble chitosan can encapsulate a gene therein to form a water-soluble chitosan-gene complex which can be feasibly introduced into cells.

Preferable is water-soluble chitosan having free amine groups, with a molecular weight of 500˜100,000 Da. More preferably, the water-soluble chitosan ranges in molecular weight from 1,000 to 50,000 Da.

Useful is, for example, the water-soluble chitosan which can be prepared by treating an organic or inorganic acid salt solution of chitosan oligosaccharide solution with trialkyl amine, 2) adding an organic solvent to the solution to remove the organic acid or inorganic acid linked with chitosan in the form of a trialkyl amine salt and recovering chitosan oligosaccharide free of organic or inorganic salt, treating the acid-free chitosan oligosaccharide solution with an inorganic acid, followed by purification, as disclosed in Korean Pat. No. 441,270, issued to the present inventors.

For effective gene delivery, the folic acid-conjugated, low-molecular weight, water-soluble chitosan nanoparticles range in size from 50 to 250 nm and more preferably from 50 to 150 nm. The nanoparticles in the range can enter the endosomes so as to act as a gene carrier [NAH, Jae-Woon et al., J. of Cont. ReL., 78, 273-284 (2002)].

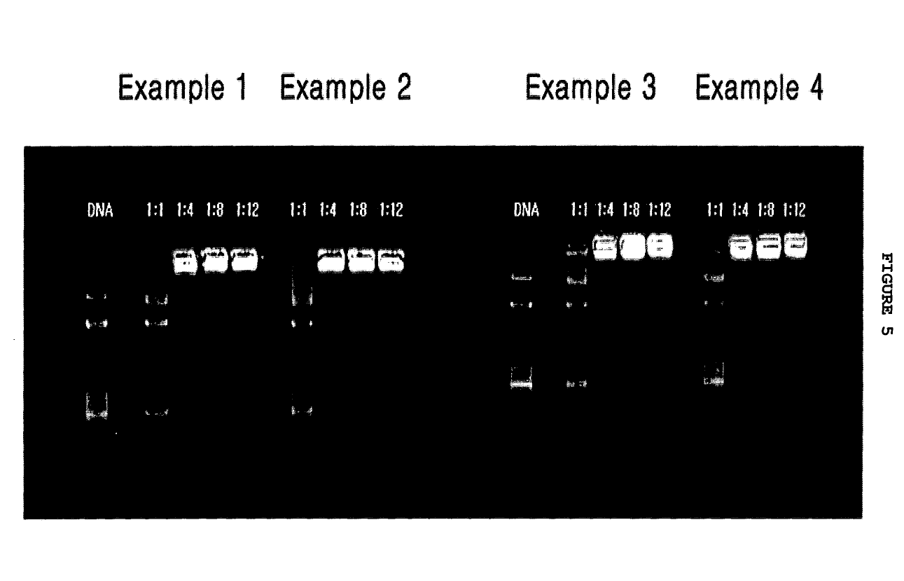

When the water-soluble chitosan nanoparticles and the gene are mixed in a weight ratio of 1:2˜1:50, the water-soluble chitosan nanoparticle-gene complex can be effectively formed. The complex formed from the components in this range was found to have low mobility upon electrophoresis, which demonstrates that the components in the range form a complete complex, effective in gene delivery (FIG. 5).

In accordance with another aspect thereof, the present invention provides a method of preparing a water-soluble chitosan nanoparticle for gene delivery, comprising, as represented by the following Reaction Formula 1, linking a low-molecular weight, water-soluble chitosan (3) to a folic acid (2) via an amide bond to form the conjugate compound of Chemical Formula 1

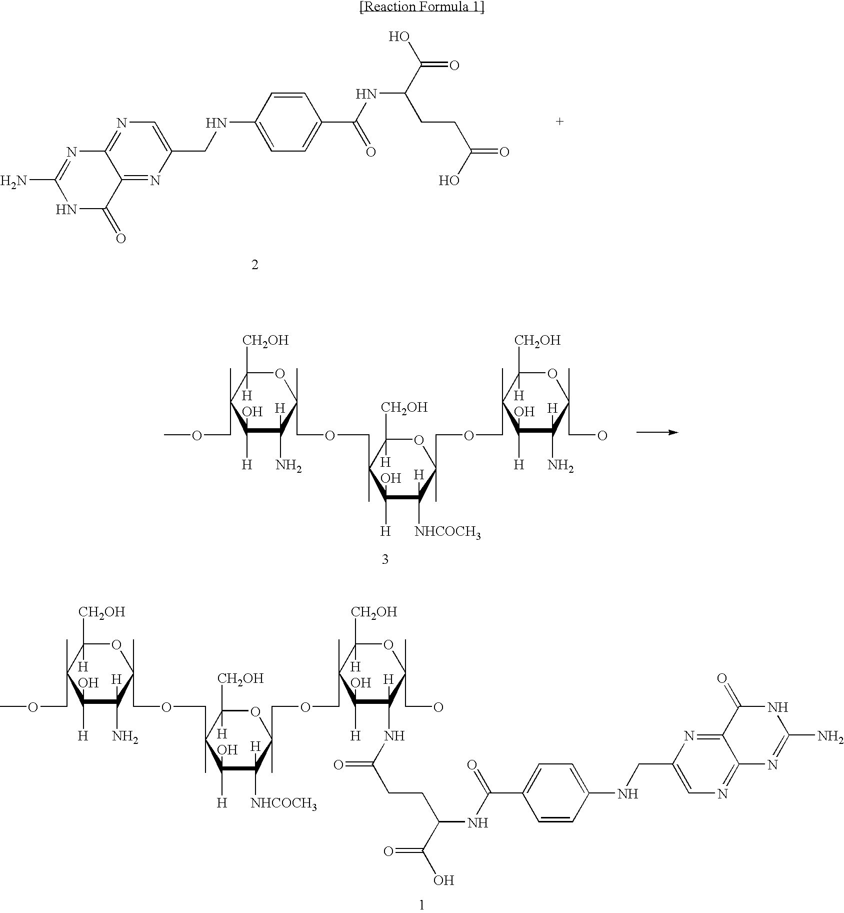

A detailed description is given of the method below.

First, a low-molecular weight, water-soluble chitosan (3) is dissolved in DMSO (dimethyl sulfoxide) to give a low-molecular weight, water-soluble chitosan. In greater detail, a suitable amount of low-molecular weight, water-soluble chitosan (3) is dissolved in distilled water and DMSO is added thereto with stirring, to give a low-molecular weight, water-soluble chitosan solution. The low-molecular weight, water-soluble chitosan (3) useful in the present invention may be prepared by the method disclosed in Korean Pat. No. 441,270, issued to the present inventors. Preferable for effective gene delivery is water-soluble chitosan (3) ranging in molecular weight from 500 to 100,000 Da and more preferably from 1,000 to 50,000 Da.

Thereafter, a solution of folic acid (2) in EDC (1-ethyl-(3-3-dimethyl aminopropyl)carbodiimide hydrochloride) is prepared. In this regard, the solution can be prepared by adding folic acid and EDC to DMSO. The folic acid is used in a comparable molar ratio with the low-molecular weight, water-soluble chitosan while the amount of EDC is preferably 1.2 times as large as that of folic acid. This process is preferably carried out in a dark room because folic acid may undergo photodegradation.

Afterwards, the folic acid solution and the low-molecular weight, water-soluble chitosan solution are mixed with stirring to prepare low-molecular weight, water-soluble chitosan nanoparticles for gene delivery in accordance with the present invention.

The mixing can be conducted in such a manner that the folic acid solution is dropwise added while the low-molecular weight, water-soluble chitosan solution is stirred. Preferably, the low-molecular weight, water-soluble chitosan solution and the folic acid solution are mixed in a weight ratio of 90˜110:0.5˜1.5. The mixing process is preferably conducted in a light-tight room lest folic acid be degraded by light.

As for the stirring, it is implemented for an appropriate time period such that the resulting folic acid-conjugated low-molecular weight, water-soluble chitosan nanoparticles are not destroyed by physical force. Preferably, stirring is conducted for 10˜15 hours.

Optionally, the method according to the present invention may comprise dialyzing and freeze-drying steps.

To remove by-products therefrom, the nanoparticles are dialyzed against distilled water for 3˜5 days, followed by freeze-drying. Freeze-drying may be carried out using a typical freeze-dryer or in a typical process. As a result, folic acid-conjugated, low-molecular weight, water-soluble chitosan nanoparticles free of by-products can be obtained in a solid state.

With a size ranging from 50 to 250 nm, the folic acid-conjugated, low-molecular weight, water-soluble chitosan nanoparticles can readily enter endosomes so as to act as a gene carrier.

In accordance with a further aspect thereof, the present invention provides a method of preparing a water-soluble chitosan-gene complex. This method features the encapsulation of a gene within the low-molecular weight, water-soluble chitosan nanoparticles prepared according to the present invention.

In the complex, the gene and the water-soluble chitosan nanoparticles are preferably present in a weight ratio of 1:2˜1:50. Water-soluble chitosan nanoparticles that are effective as gene carriers range in size from 50 nm to 250 nm.

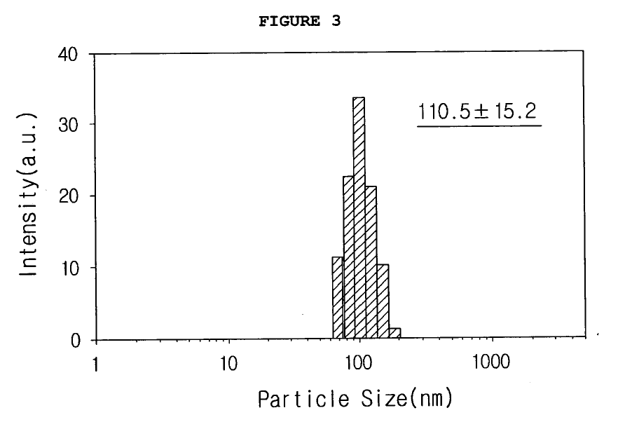

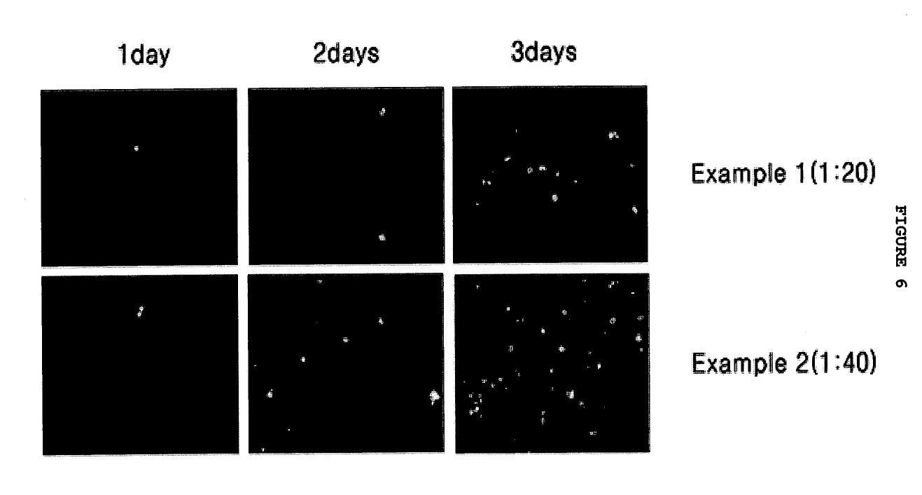

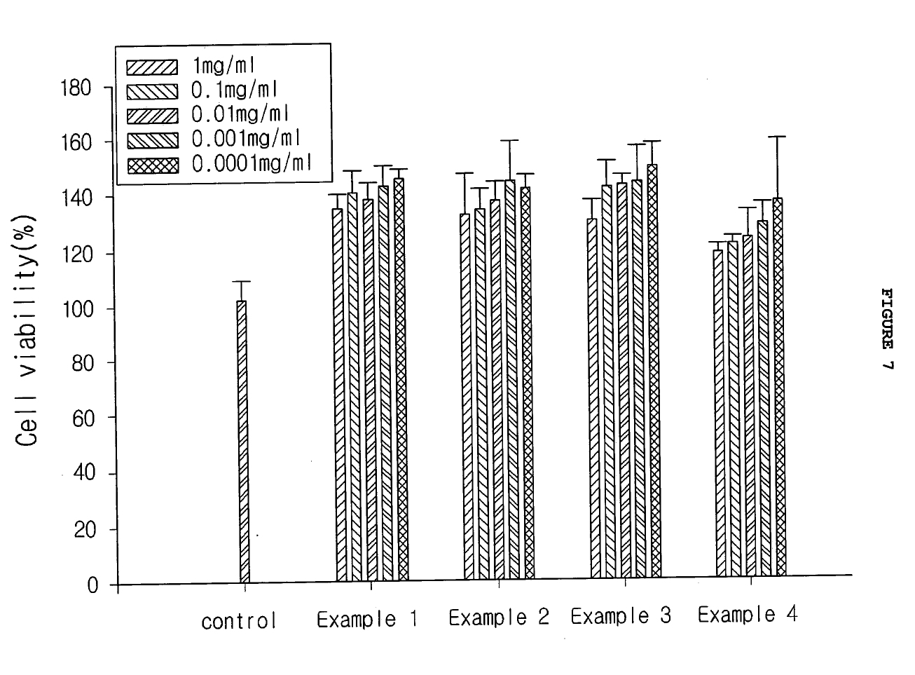

The folic acid-conjugated, low-molecular weight, water-soluble chitosan nanoparticles are simple to prepare since the strong reactivity of the low-molecular weight, water-soluble chitosan allows folic acid to be readily introduced thereinto. In addition, the folic acid-conjugated, low-molecular weight, water-soluble chitosan nanoparticles are of low or zero-toxicity (FIG. 7), have sizes suitable for use as gene carriers (FIG. 3, 4), can readily form complexes with DNA (FIG. 5), allow high gene expression rates (FIG. 6), and are excellent in targeting tumor cells which are rich in folic acid receptors. Consequently, the folic acid-conjugated, low-molecular weight, water-soluble chitosan nanoparticles can be useful as gene carriers.

### MODE FOR INVENTION

A better understanding of the present invention may be obtained through the following examples which are set forth to illustrate, but are not to be construed as the limit of the present invention.

### Example 1

### Preparation of Nanoparticles of Low-Molecular Weight, Water-Soluble Chitosan Conjugated with Folic Acid (LMWSC-FA)

Low-molecular, water-soluble chitosan (LMWSC), having a molecular weight of 10,000 Da and a degree of deacetylation of 97%, was supplied from KITTOLIFE Co. Ltd. The folic acid (FA) used in this example was approx. 98% pure. EDC was purchased from Sigma Chemical Co (Mw 191.7). All other chemical reagents were of the highest obtainable quality and were used without further purification.

In 1 ml of distilled water was dissolved 50 ml of LMWSC, having a molecular weight of 10,000 Da, and then 10 ml of DMSO was added thereto, followed by stirring at room temperature.

Folic acid was added in an amount of 3 mol % based on 50 mg of the LMWSC to 2 ml of DMSO in a dark room at room temperature, and was diluted with an EDC solution in an amount at a 1:1.2 mole ratio of the folic acid to give a folic acid solution.

Thereafter, the folic acid solution was slowly added to the low-molecular, water-soluble chitosan solution and then stirred overnight at room temperature in a dark room. Under a dark condition, the reactant solution was dialyzed against distilled water for 4 days, followed by freeze-drying for 3 days in a freeze dryer (77510-03, LABCONCO, USA) to produce 40-60 mg of folic acid-conjugated, low-molecular weight, water-soluble chitosan nanoparticles (70˜80%).

### Examples 2 to 4

### Preparation of Folic Acid-Conjugated, Low-Molecular Weight, Water-Soluble Chitosan Nanoparticle

Folic acid-conjugated, low-molecular weight, water-soluble chitosan nanoparticles were prepared in the same manner as above, with the exception that folic acid was used in amounts of 5 mol %, 10 mol % and 15 mol % based on 50 mg of the LMWSC.

### Example 3

### Preparation of Folic Acid-Conjugated, Low-Molecular Weight, Water-Soluble Chitosan Gene Complex

To a cell was added sterile water, followed by the folic acid-conjugated, low-molecular weight, water-soluble chitosan nanoparticles prepared in Examples 1 to 4. A DNA molecule pEGFP-N1 (Clontech, Palo Alto, Calif.) was added to form a total volume of 20 at. After shaking for 30 seconds, the solution was allowed to stand for 30 min at 4° C. to form a complex. In this regard, the weight ratio of the DNA molecule to the folic acid-conjugated, low-molecular weight, water-soluble chitosan nanoparticles was 1:4.

### Examples 4 to 7

### Preparation of Folic Acid-Conjugated, Low-Molecular Weight, Water-Soluble Chitosan Gene Complex

The same procedure as in Example 3 was carried out, with the exception that the weight ratios of the DNA molecule and the folic acid-conjugated, low-molecular weight, water-soluble chitosan nanoparticles were respectively 1:8, 1:12, 1:20 or 1:40.

### Experimental Example 1

### FT-IR Spectroscopy of Folic Acid-Conjugated, Low-Molecular Weight, Water-Soluble Chitosan Nanoparticles

To examine the structural states of the folic acid-conjugated, low-molecular weight, water-soluble chitosan nanoparticles prepared in Examples 1 to 4, an experiment was carried out with an FT-IR spectrometer as follows.

Mixtures of 100:1 the compounds of Examples 1 to 4 to KBr were stirred for 10 min and then dehydrated at 60 C for 12 hours in a vacuum to give samples.

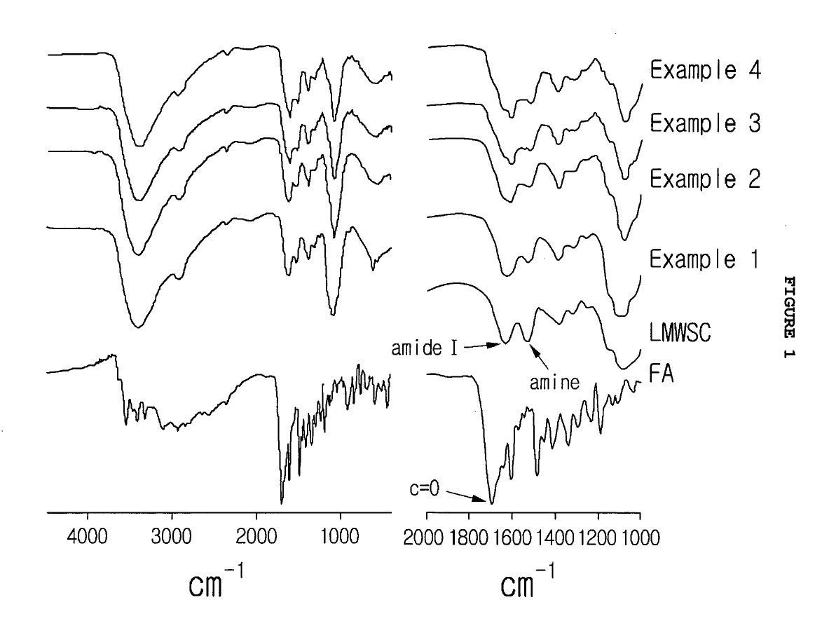

These samples were analyzed to determine the structures thereof using an FT-IR spectrometer (Shimadzu, FR—IR 8700, Japan), and the results are shown in FIG. 1.

As shown in FIG. 1, an increase in the amount of folic acid induced a decrease in the amine peak at 1550 cm⁻¹, but also induced an increase in the amide I absorption peak, indicating that the carboxylic group of folic acid forms an amide bond with the amine group of the low-molecular weight, water-soluble chitosan nanoparticles.

Accordingly, the spectra of FIG. 1 demonstrate that the compounds prepared in Examples 1 to 4 are folic acid-conjugated, low-molecular weight, water-soluble chitosan nanoparticles in which folic acid is effectively grafted to the free amine groups of the low-molecular weight, water-soluble chitosan.

### Experimental Example 2

### ¹H NMR Spectroscopy and Quantitative Analysis of Folic Acid-Conjugated, Low-Molecular Weight, Water-Soluble Chitosan Nanoparticles

To examine the structural states of the folic acid-conjugated, low-molecular weight, water-soluble chitosan nanoparticles prepared in Examples 1 to 4, the following experiment was carried out with an ¹H NMR spectrometer.

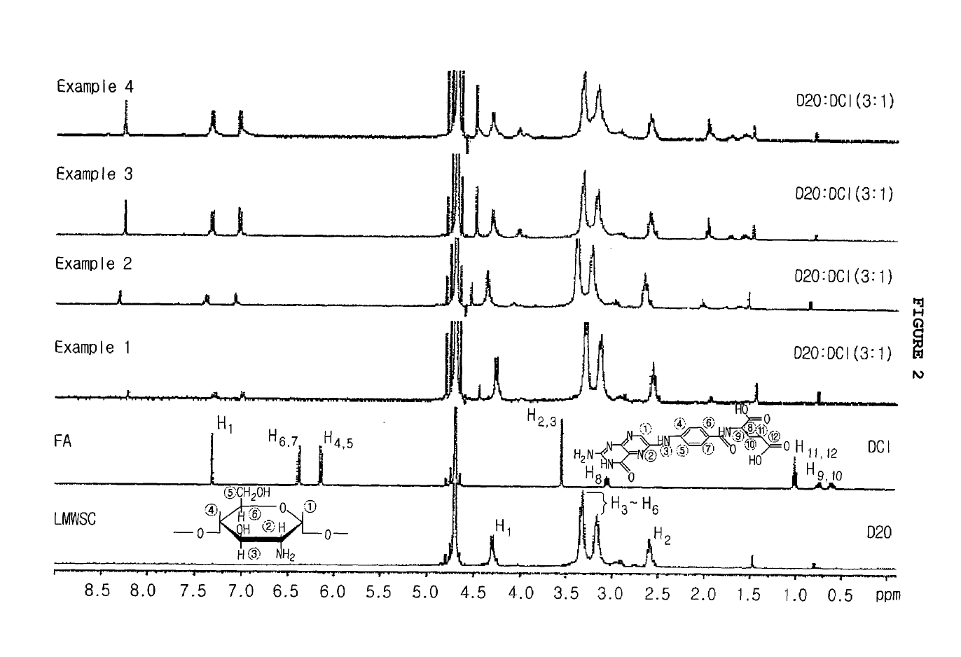

The compounds of Examples 1 to 4 were dissolved in a mixture solvent of D₂O and DCL (D₂O:DCL=3:1 v/v) and analyzed using a ¹H NMR spectrometer (AVANCE 400 (400 MHz), Bruker, Germany) at 298K. The spectrum results are shown in FIG. 2.

Based on the ¹H NMR spectrum data, the degree of deacetylation (DDA (%)) and the degree of substitution of (DS (%)) of folic acid were calculated for the low-molecular weight, water-soluble chitosan nanoparticles as follows.

DDA (%)=1−(hydrogen area of acetamide at 1.5 ppm/hydrogen area of chitosan nanoparticles at 2.5 ppm)

DS (%)=(hydrogen area of acetamide at 1.5 ppm/hydrogen area of folic acid at 8.4 ppm)

$$DDA ( \% ) = ( 1 - \frac{\begin{matrix}hydrogen area of \\ acetamide at 1.5 ppm\end{matrix}}{\begin{matrix}hydrogen area of chitosan \\ nanoparticles at 2.5 ppm\end{matrix}} ) \times 100$$

$$DS ( \% ) = ( \frac{hydrogen area of acetamide at 1.5 ppm}{hydrogen area of folic acid at 8.4 ppm} ) \times 100$$

The calculations of DDA and DS are given in Table 1, below.

TABLE 1

| Example Nos. | DDA(%) | DS(%) |
|---|---|---|
| 1 | 96.38 | 2.48 |
| 2 | 96.29 | 4.20 |
| 3 | 96.33 | 9.22 |
| 4 | 96.12 | 13.74 |

As seen in the ¹H NMR spectra of FIG. 2 for the low-molecular water-soluble chitosan compounds of Examples 1 to 4, hydrogen atoms of carbon 1 and 2 of the chitosan appeared at 4.3 ppm and 2.6 ppm, respectively, while hydrogen atoms of carbon 3 to 6 of the chitosan were identified at 3.4˜3.1 ppm. As for the folic acid, its hydrogen atoms were identified by peaks at 7.3 ppm for carbon 1, at 3.6 ppm for carbon 2 and 3, at 6.1 ppm for carbon 4 and 5, at 6.4 ppm for carbon 6 and 7, and at 3.1 ppm for carbon 8. From these spectrum data, it is apparent that the compounds prepared in Examples 1 to 4 are low-molecular weight, water-soluble chitosan nanoparticles conjugated with folic acid.

Also, the data of Table 1 shows an increase in DS with the amount of folic acid increasing, demonstrating that folic acid was effectively grafted to free amine groups of the low-molecular weight, water-soluble chitosan.

### Experimental Example 3

### Measurement of Folic Acid-Conjugated, Low-Molecular Weight, Water-Soluble Chitosan Nanoparticles for Size and Morphology

The folic acid-conjugated, low-molecular weight, water-soluble chitosan nanoparticles prepared in Examples 1 to 4 were measured for size and morphology as follows.

Freeze-dried, folic acid-conjugated, low-molecular weight, water-soluble chitosan nanoparticles were dispersed at a concentration of 1 mg/ml in distilled water, followed by the determination of particle size using ELS-8000 (Otsuka, Electronics, Japan) based on dynamic light scattering.

Then, the folic acid-conjugated, low-molecular weight, water-soluble chitosan nanoparticles were observed for size and morphology using a TEM (Transmission Electron Microscope; JEOL JEM-2000 FX-II).

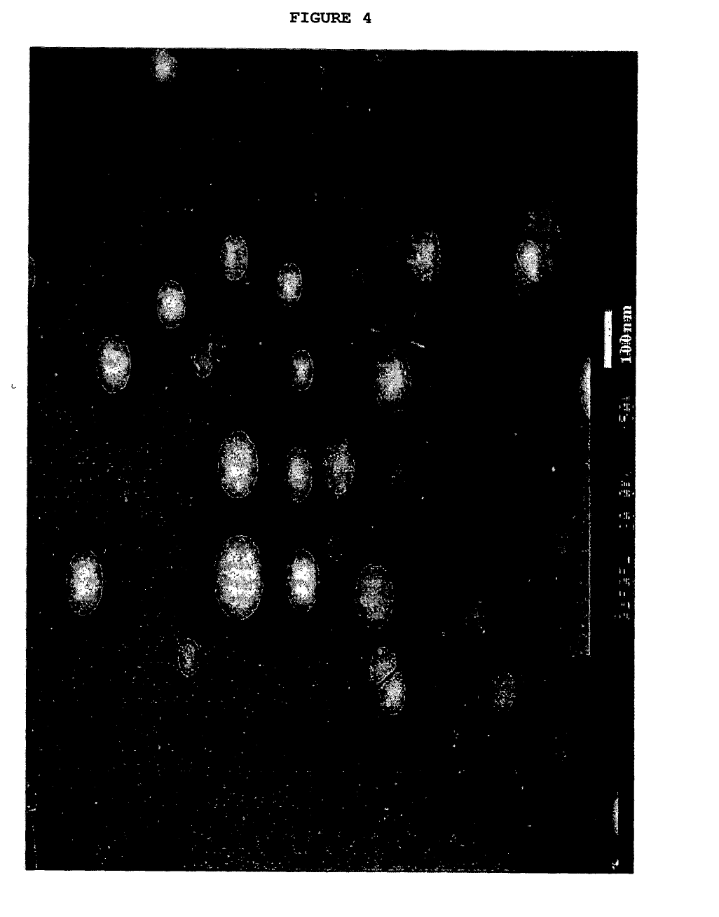

The measurements and observations are given in FIGS. 3 and 4.

As graphed in FIG. 3, the folic acid-conjugated, low-molecular weight, water-soluble chitosan nanoparticles have a mean size of 110 nm with a very narrow size distribution. As identified in the photograph of FIG. 4, the folic acid-conjugated, low-molecular weight, water-soluble chitosan nanoparticles are globular in shape, with a size of approximately 100 nm, which is coincident with the dynamic light scattering measurement. As a result, the nanoparticles were observed to have a size suitable for use as gene carriers.

### Experimental Example 4

### Identification of Folic Acid-Conjugated, Low-Molecular Weight, Water-Soluble Chitosan-Nanoparticle-Gene Complex

To determine whether the folic acid-conjugated, low-molecular weight, water-soluble chitosan nanoparticles can play a role as a gene carrier, they were examined for their ability to associate with a DNA molecule to form a gene complex as follows.

pEGFP-N1 (Clontech, Palo Alto, Calif.) was mixed in weight ratios of 1:1, 1:4, 1:8 and 1:12 with the folic acid-conjugated, low-molecular weight, water-soluble chitosan nanoparticles prepared in Examples 1 to 4, respectively, to form folic acid-conjugated, low-molecular weight, water-soluble chitosan nanoparticle-gene complexes.

The formation of the nanoparticle-gene complexes were identified by electrophoresis, which was carried out on 1% agarose gel for 30 min in the presence of an electric field of 100 V. The results are photographed as shown in FIG. 5.

The plasmid DNA, as seen in the photographs, migrated normally when it was left naked, but showed very slow mobility after it was mixed with the nanoparticles in the above-mentioned weight ratios, except for a weight ratio of 1:1.

Hence, it was found that the folic acid-conjugated, low-molecular weight, water-soluble chitosan nanoparticles can form complexes with DNA molecules and are useful as gene carriers.

### Experimental Example 5

### Assay of Folic Acid-Conjugated, Low-Molecular Weight, Water-Soluble Chitosan Nanoparticles for Gene Expression Efficiency

The folic acid-conjugated, low-molecular weight, water-soluble chitosan nanoparticle-gene complexes according to the present invention were assayed for gene expression efficiency in cells as follows.

HEK-293 cells were cultured in a DMEM (Dulbecco s Modified Eagle Medium) supplemented with 10% FBS (fetal bovine serum) and an antibiotic at 37 C in a 5% CO₂ incubator. Thereafter, the cells were seeded at a density of 4 10⁴ cells/well into 24-well plates, and were incubated for 24 hours.

The plates were separated into a pH 6.2 group and a pH 7.0 group before replacement with DMEM(+) media. Then, the complex of Example 6 or 7 was added to each well and incubated for 4 hours, followed by replacing the medium in the pH 6.2 wells with pH 7.0 DMEM(+). The gene expression was monitored for 3 days under a fluorescence microscope (Olympus IX 71, Olympus, Japan) and the results are shown in FIG. 6.

The gene expression rates were found to increase with time, as seen in the photographs of FIG. 6.

### Experimental Example 6

### MTT Assay

In order to examine the cytotoxicity of the folic acid-conjugated, low-molecular weight, water-soluble chitosan nanoparticles according to the present invention, an MTT assay was conducted as follows.

HEK-293 cells were seeded at a density of 1 10⁴ cells/well into 96-well plates and cultured overnight at 37 C in a 5% CO₂ incubator. The folic acid-conjugated, low-molecular weight, water-soluble chitosan nanoparticles of Examples 1 to 4 were added in amounts such that they formed final nanoparticle concentrations of 1, 0.1, 0.01, 0.001 and 0.0001 mg/ml to each well. MTT assay was conduced at intervals of 2 days, 3 days and 5 days.

After the addition of 50 L of a 3 mg/ml MTT solution to each well, the cells were incubated at 37 C for 4 hours. The supernatant was completely aspirated before DMSO was added in an amount of 100 L to each well. 10 min incubation was followed by reading the optical density in a microplate reader (VERSA MAX). Cell viability was calculated using the following equation.

$$Cell Viability ( \% ) = \frac{OD 570 of Sample}{OD 570 of Control} \times 100$$

The OD570 of sample and the OD570 of control represent the absorbances at 570 nm, measured from wells in which the cells are treated with the folic acid-conjugated, low-molecular weight, water-soluble chitosan nanoparticles and with PBS alone, respectively.

The results are graphed in FIG. 7.

As seen in the graph of FIG. 7, higher cell viability was obtained when the cells were treated with the folic acid-conjugated, low-molecular weight, water-soluble chitosan nanoparticles of Examples 1 to 4 than when treated with PBS alone. Therefore, the folic acid-conjugated, low-molecular weight, water-soluble chitosan nanoparticles according to the present invention are not toxic to cells.

### INDUSTRIAL APPLICABILITY

As described hitherto, the folic acid-conjugated, low-molecular weight, water-soluble chitosan nanoparticles are simple to prepare since the strong reactivity of the low-molecular weight, water-soluble chitosan allows folic acid to be readily introduced thereinto. In addition, the folic acid-conjugated, low-molecular weight, water-soluble chitosan nanoparticles are of low or zero-toxicity, have sizes suitable for use as gene carriers, can readily form complexes with DNA, allow high gene expression rates, and are excellent in targeting tumor cells which are rich in folic acid receptors. Consequently, the folic acid-conjugated, low-molecular weight, water-soluble chitosan nanoparticles can be useful as gene carriers.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A conjugate compound of low-molecular weight, water-soluble chitosan and folic acid, represented by the following Chemical Formula 1,

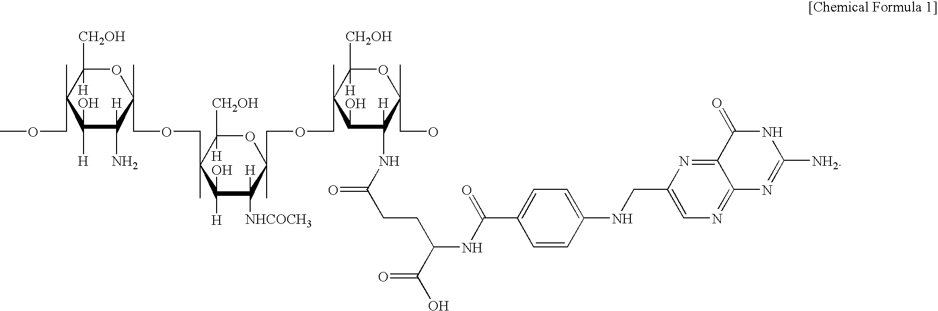

2. A water-soluble chitosan nanopalticle for gene delivery, comprising the conjugate compound of low-molecular weight, water-soluble chitosan and folic acid as set forth in claim 1.

3. The water-soluble chitosan nanoparticle as set forth in claim 2, wherein low-molecular weight, water-soluble chitosan and the folic acid are present in a weight ratio of 90˜110:0.5˜1.5.

4. The water-soluble chitosan nanoparticle as set forth in claim 2, wherein the gene is a DNA molecule.

5. A water-soluble chitosan-gene complex, comprising the water-soluble chitosan nanoparticle as set forth in claim 2 and a gene encapsulated therein, wherein said nanoparticle forms an aggregate with a hydrophobic core surrounded by a hydrophilic shell in an aqueous solution.

6. The water-soluble chitosan-gene complex as set forth in claim 5, wherein the water-soluble chitosan nanoparticle and the gene are present in a weight ratio of 1:2˜1:50.

7. The water-soluble chitosan-gene complex as set forth in claim 5, wherein the gene is a DNA molecule.

8. A method of preparing the water-soluble chitosan nanoparticle of claim 2 for gene delivery, comprising, reacting a low-molecular weight, water-soluble chitosan of Chemical Formula 3 with folic acid of Chemical Formula 2 in a solvent to form a conjugate compound of Chemical Formula 1,

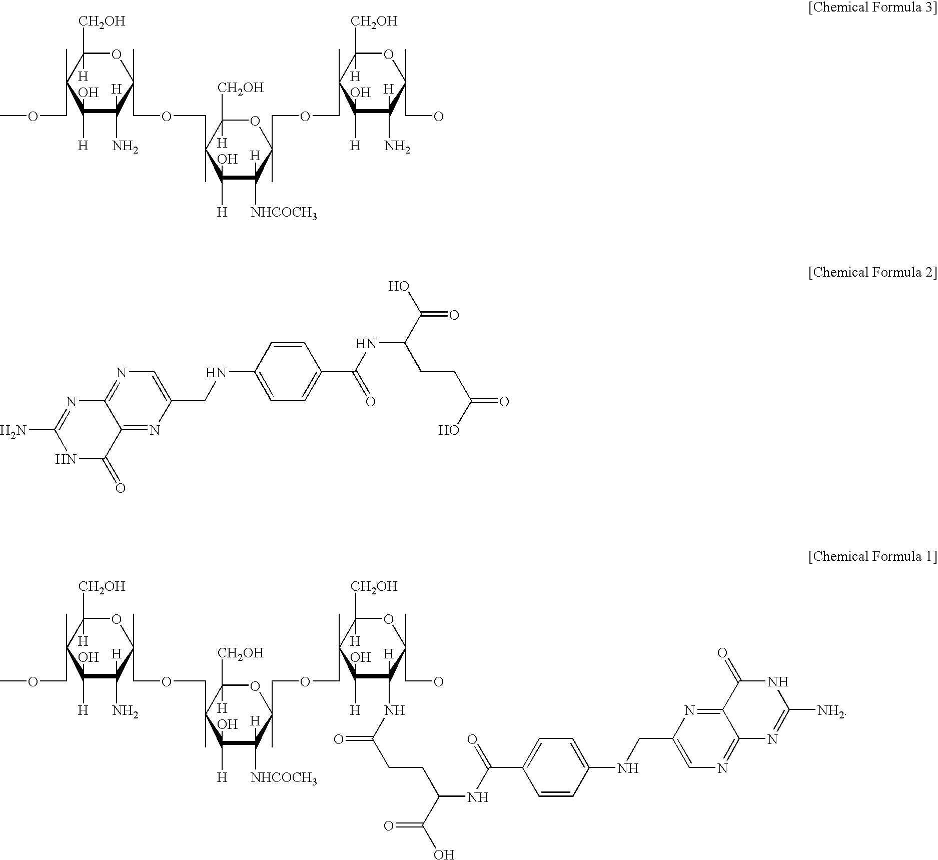

9. The method as set forth in claim 8, wherein the low-molecular weight, water-soluble chitosan and folic acid are reacted in the presence of EDC (1-ethyl-(3,3-dimethyl aminopropyl)carbodiimide hydrochloride) and DMSO as the solvent.

10. The method as set forth in claim 9, wherein the folic acid and EDC are present in 1:1.2 mole ratio.

11. The method as set forth in claim 8, wherein the low-molecular weight, water-soluble chitosan and the folic acid are present in a weight ratio of 90˜110:0.5˜1.5.

12. The method as set forth in claim 8, further comprising dialyzing and freeze-drying the conjugate compound.

13. The method as set forth in claim 8, wherein the low-molecular weight, water-soluble chitosan and folic acid are reacted in a dark room.

14. The method as set forth in claim 8, wherein the water-soluble chitosan nanoparticle for gene delivery ranges in size from 50 nm to 250 nm.

15. A method of preparing a water-soluble chitosan-gene complex, comprising encapsulating a gene in a water-soluble chitosan nanoparticle, prepared by the method as set forth in claim 8, for gene delivery.

16. The method as set forth in claim 15, wherein the water-soluble chitosan nanoparticle and the gene are present in a weight ratio of 1:2˜1:50.

17. The method as set forth in claim 15, wherein the gene is a DNA molecule.

18. The method as set forth in claim 15, wherein the water-soluble chitosan nanoparticle for gene delivery ranges in size from 50 nm to 250 nm.
